(19) United States
(12) Patent Application Publication
(10) Pub. No.: US 2022/0218230 A1
(43) Pub. Date: Jul. 14, 2022

(54) SYSTEM AND METHOD OF DETECTING WALKING ACTIVITY USING WAIST-WORN INERTIAL SENSORS

(71) Applicant: Robert Bosch GmbH, Stuttgart (DE)

(72) Inventors: Huan Song, San Jose, CA (US); Lincan Zou, San Jose, CA (US); Liu Ren, Saratoga, CA (US)

(21) Appl. No.: 17/147,588

(22) Filed: Jan. 13, 2021

(51) Int. Cl. A61B 5/11; H04B 1/3827; A63B 24/00; A61B 5/00; G16H 20/30

(52) U.S. Cl. CPC A61B 5/112; H04B 1/385; A61B 5/1118; A63B 24/0062; A61B 5/725; A61B 5/6823; H04B 2001/3855; A61B 2562/0219; A63B 2220/803; A63B 2220/836; G16H 20/30

(57) ABSTRACT

A system and method for monitoring a walking activity are disclosed, which have three major components: a pre-processing phase, a step detection phase, and a filtering and post-processing phase. In the preprocessing phase, recorded motion data is received, reoriented with respect to gravity, and low-pass filtered. Next, in the step detection phase, walking step candidates are detected from vertical acceleration peaks and valleys resulting from heel strikes. Finally, in the filtering and post-processing phase, false positive steps are filtered out using a composite of criteria, including time, similarity, and horizontal motion variation. The method 200 is advantageously able to detect most walking activities with accurate time boundaries, while maintaining very low false positive rate.

### FIELD

The device and method disclosed in this document relates to human motion sensing and, more particularly, to detecting walking activity using waist-worm inertial sensors.

### BACKGROUND

Unless otherwise indicated herein, the materials described in this section are not admitted to be the prior art by inclusion in this section.

In recent years, wearable inertial measurement unit (IMU) sensors have been used in various domains for consumers and industries: healthcare, manufacturing, fitness tracking, entertainment, etc. Particularly, IMU sensors have been frequently incorporated into smartphones, smart watches, and smart bands for motion recording and analysis. Among the many applications for wearable IMU sensors, it is of particular interest to monitor the activity of walking. However, conventional techniques for monitoring walking activities are often prone to significant errors and are best suited for consumer applications, such as fitness tracking, in which a very high degree of accuracy is less important. What is needed is a method for monitoring walking activities that provides the higher degree of accuracy required for a broader set of commercial or industrial applications.

### SUMMARY

A method for recognizing a walking activity is disclosed. The method comprises receiving, with a processor, motion data at least including a time series of acceleration values corresponding to motions of a human that include walking. The method further comprises defining, with the processor, a first plurality of segments of the received motion data by detecting local peaks and local valleys in the time series of acceleration values, each segment in the first plurality of segments including respective motion data corresponding to an individual step of the human. The method further comprises defining, with the processor, a second plurality of segments of the received motion data by merging each of a plurality of groups of segments in the first plurality of segments, each segment in the second plurality of segments including respective motion data corresponding to a period of continuous walking by human.

A system for recognizing a walking activity is disclosed. The system comprises at least one motion sensor configured to capture motion data at least including a time series of acceleration values corresponding to motions of a human that include walking. The system further comprises a processing system having at least one processor. The at least one processor is configured to receive the motion data from the motion sensor. The at least one processor is further configured to define a first plurality of segments of the received motion data by detecting local peaks and local valleys in the time series of acceleration values, each segment in the first plurality of segments including respective motion data corresponding to an individual step of the human. The at least one processor is further configured to define a second plurality of segments of the received motion data by merging each of a plurality of groups of segments in the first plurality of segments, each segment in the second plurality of segments including respective motion data corresponding to a period of continuous walking by human.

A non-transitory computer-readable medium for recognizing a walking activity is disclose. The computer-readable medium stores program instructions that, when executed by a processor, cause the processor to receive motion data at least including a time series of acceleration values corresponding to motions of a human that include walking. The program instructions, when executed by a processor, further cause the processor to define a first plurality of segments of the received motion data by detecting local peaks and local valleys in the time series of acceleration values, each segment in the first plurality of segments including respective motion data corresponding to an individual step of the human. The program instructions, when executed by a processor, further cause the processor to define a second plurality of segments of the received motion data by merging each of a plurality of groups of segments in the first plurality of segments, each segment in the second plurality of segments including respective motion data corresponding to a period of continuous walking by human.

### BRIEF DESCRIPTION OF THE DRAWINGS

The foregoing aspects and other features of the system and method are explained in the following description, taken in connection with the accompanying drawings.

FIG. 1 shows a system for monitoring a walking activity.

FIG. 2 shows a flow diagram for a method for monitoring a walking activity.

### DETAILED DESCRIPTION

For the purposes of promoting an understanding of the principles of the disclosure, reference will now be made to the embodiments illustrated in the drawings and described in the following written specification. It is understood that no limitation to the scope of the disclosure is thereby intended. It is further understood that the present disclosure includes any alterations and modifications to the illustrated embodiments and includes further applications of the principles of the disclosure as would normally occur to one skilled in the art which this disclosure pertains.

### System Overview

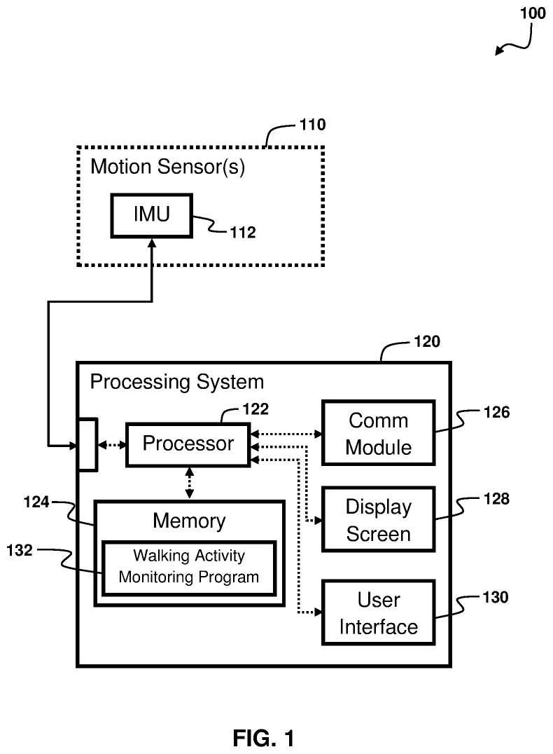

FIG. 1 shows a system 100 for monitoring a walking activity. The system 100 at least comprises motion sensors 110 and a processing system 120. The motion sensors 110 include one or more sensors configured to measure or track motions corresponding to a walking activity. The processing system 120 is configured to process motion data received from the motion sensors 110 to recognize and segment continuous walking regions in the motion data. By accurately recognizing walking and segmenting continuous walking regions, the system 100 can provide fundamental information about human activities and enable important subsequent functionalities including step count, path estimation, gait recognition, and indoor localization, etc. When utilized in a smart manufacturing context, walking recognition has significant advantageous use cases. For example, at the workstations of an assembly line, walking activity of the operator often indicates some non-ideal setup causes wasted operation time. Understanding when and where they occur is the basis to potentially optimize the operational procedure in order to improve efficiency and reduce fatigue of operators. In such scenarios, the system 100 can provide a cost-efficient and scalable approach to continuously record all motions, and enable optimizations of the assembly line.

The motion sensors 110 comprise at least one sensor configured to track the motions that comprise the walking activity. In at least some embodiments, the motion sensors 110 comprise at least one inertial measurement unit (IMU) 112. The IMU 112 includes, for example, one or more accelerometers, one or more gyroscopes, and one or more magnetometers configured to provide motion data in the form of acceleration measurements, orientation measurements, and magnetic field measurements. In one embodiment, the IMU 112 comprises an integrated 9-degrees-of-freedom (9-DOF) inertial sensor that provides triaxial acceleration measurements, triaxial gyroscopic/orientation measurements, and triaxial magnetic field measurements.

In at least one embodiment, the motion sensors 110 and/or the IMU 112 is worn on the body of a human, for example, on the wait, back, chest, or hip of the human. It will be appreciated that these locations on the human body will tend to result in more stable motion data compared to wrist or hand worn sensors. However, the techniques described herein do not necessarily exclude the usage of wrist or hand worn sensors. In some embodiments, the IMU 112 may be integrated with an object that is carried (rather than worn) by the human, such as a smartphone that is carried by the human in his or her pocket. In at least one embodiment, the motion sensors 110 are integrated with the processing system 120 in a single device, such as a smartphone or a similar device. However, in alternative embodiments, the motion sensors 110 are independent of the processing system 120 and communicate motion data to the processing system 120 by a wired or wireless data connection.

The processing system 120 is configured to process motion data captured by the motion sensors 110 to recognize and segment continuous walking regions. Particularly, the processing system 120 is configured to detect time regions of the motion data that correspond to individual steps and/or correspond to continuous periods of walking. To this end, the processing system 120 generates labels or timestamps indicating the times at which regions of continuous walking begin and end. In some embodiments, the processing system 120 further determines secondary metadata based on the labeled walking regions of the motion data, such as step count, path estimation, gait recognition, and indoor localization, etc.

In the illustrated exemplary embodiment, the processing system 120 comprises at least one processor 122, at least one memory 124, a communication module 126, a display screen 128, and a user interface 130. However, it will be appreciated that the components of the processing system 120 shown and described are merely exemplary and that the processing system 120 may comprise any alternative configuration. Particularly, the processing system 120 may comprise any computing device such as a smart watch, a smart phone, a tablet computer, desktop computer, a laptop computer, or another electronic device. Thus, the processing system 120 may comprise any hardware components conventionally included in such computing devices. As noted above, the motion sensors 110 may be integrated with the processing system 120 as a single device. However, in other embodiments, the processing system 120 is independent from the motion sensors 110 and may perform processing for a plurality of separate motion sensors 110 associated with a plurality of different individual humans.

The memory 124 is configured to store data and program instructions that, when executed by the at least one processor 122, enable the processing system 120 to perform various operations described herein. The memory 124 may be of any type of device capable of storing information accessible by the at least one processor 122, such as a memory card, ROM, RAM, hard drives, discs, flash memory, or any of various other computer-readable medium serving as data storage devices, as will be recognized by those of ordinary skill in the art. Additionally, it will be recognized by those of ordinary skill in the art that a “processor” includes any hardware system, hardware mechanism or hardware component that processes data, signals or other information. Thus, the at least one processor 122 may include a central processing unit, graphics processing units, multiple processing units, dedicated circuitry for achieving functionality, programmable logic, or other processing systems. Additionally, it will be appreciated that, although the processing system 120 is illustrated as single device, the processing system 120 may comprise several distinct processing systems 120 that work in concert to achieve the functionality described herein.

The communication module 126 may comprise one or more transceivers, modems, processors, memories, oscillators, antennas, or other hardware conventionally included in a communications module to enable communications with various other devices. In at least some embodiments, the communication module 126 includes a Wi-Fi module configured to enable communication with a Wi-Fi network and/or Wi-Fi router (not shown). In further embodiments, the communications module 126 may further include a Bluetooth® module, an Ethernet adapter and communications devices configured to communicate with wireless telephony networks.

The display screen 128 may comprise any of various known types of displays, such as LCD or OLED screens. In some embodiments, the display screen 128 may comprise a touch screen configured to receive touch inputs from a user. The user interface 130 may suitably include a variety of devices configured to enable local operation of the processing system 120 by a user, such as a mouse, trackpad, or other pointing device, a keyboard or other keypad, speakers, and a microphone, as will be recognized by those of ordinary skill in the art. Alternatively, in some embodiments, a user may operate the processing system 120 remotely from another computing device which is in communication therewith via the communication module 126 and has an analogous user interface.

The program instructions stored on the memory 124 include a walking activity monitoring program 132. As discussed in further detail below, the processor 122 is configured to execute the walking activity monitoring program 132 to detect time regions of the motion data that correspond to individual steps and/or correspond to continuous walking. Moreover, the processor 122 is configured to execute the walking activity monitoring program 132 to generate labels or timestamps indicating the times at which regions of continuous walking begin and end. In some embodiments, the processor 122 is configured to execute the walking activity monitoring program 132 to determine secondary metadata based on the labeled walking regions of the motion data, such as step count, path estimation, gait recognition, and indoor localization, etc.

### Method for Monitoring a Walking Activity

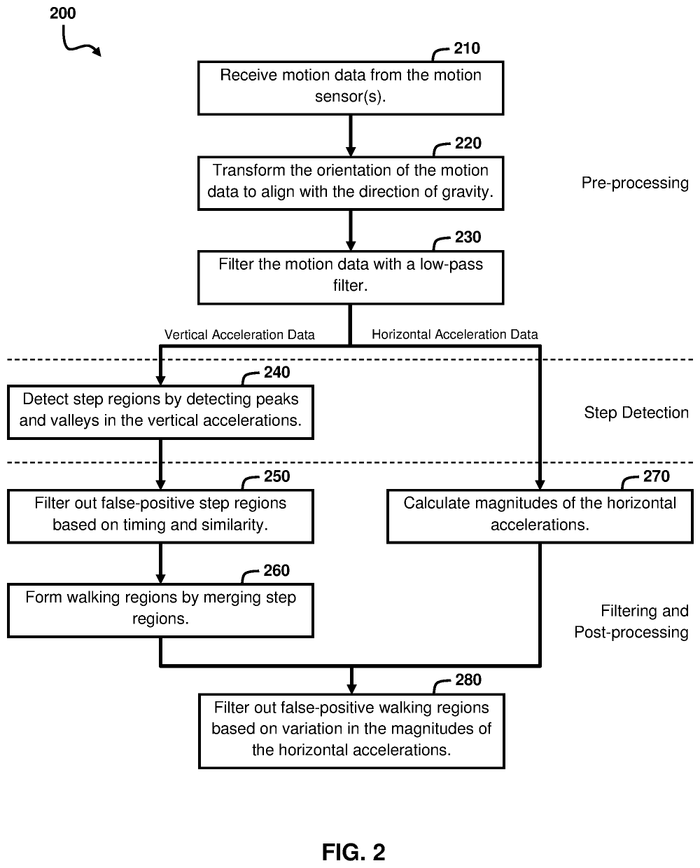

FIG. 2 shows a flow diagram for a method 200 for monitoring a walking activity. In the description of these method, statements that some task, calculation, or function is performed refers to a processor (e.g., the processor 122 of the processing system 120) executing programmed instructions stored in non-transitory computer readable storage media (e.g., the memory 124 of the processing system 120) operatively connected to the processor to manipulate data or to operate one or more components of the processing system 120 or the system 100 to perform the task or function. Additionally, the steps of the methods may be performed in any feasible chronological order, regardless of the order shown in the figures or the order in which the steps are described.

In summary, the method 200 has three major components: a pre-processing phase, a step detection phase, and a filtering and post-processing phase. In the preprocessing phase, recorded motion data is received, reoriented with respect to gravity, and low-pass filtered. Next, in the step detection phase, walking step candidates are detected from vertical acceleration peaks and valleys resulting from heel strikes. Finally, in the filtering and post-processing phase, false positive steps are filtered out using a composite of criteria, including time, similarity, and horizontal motion variation. The method 200 is advantageously able to detect most walking activities with accurate time boundaries, while maintaining very low false positive rate.

In greater detail and with continued reference to FIG. 2, the method 200 begins, in the pre-processing phase, with receiving motion data from the motion sensor(s) (block 210). Particularly, the processor 122 receives motion data corresponding to motions of the human wearing or carrying the motion sensor(s) 110 (e.g., the IMU 112), which may include motions corresponding to a walking activity. In one embodiment, the processor 122 receives a stream of motion data directly from the motion sensor(s) 110 and writes the stream of motion data to the memory 124, for example in a buffer that is implemented on the memory 124. Alternatively, some other component collects the motion data from the motion sensor(s) 110 and the processor 122 may read the motion data from the memory 124 or from some other local storage medium, or the processor 122 may operate the communication module 126 to receive the motion data from some other computing device or remote storage device.

In the case that the motion sensor(s) 110 comprise an integrated 9-DOF IMU 112, the raw motion data comprises a time series of triaxial acceleration data denoted as the vector a=[aₓ, a_{y}, a_{z}], a time series of triaxial orientation data denoted as the vector o=[oₓ, o_{y}, o_{z}], and a time series of triaxial magnetic field data denoted as the vector m=[mₓ, m_{y}, m_{z}].

The method 200 continues, in the pre-processing phase, with transforming the orientation of the motion data to align with the direction of gravity (block 220). Particularly, the processor 122 transforms the orientation of the motion data {a, o, m} to align with the direction of gravity (i.e., the world frame). It will be appreciated that the raw measurements of the motion data are generally oriented in the manner of the motion sensor(s) 110 (e.g., the IMU 112) themselves. The processor 122 calculates aligned motion data by determining the direction of gravity based on the raw acceleration data a and rotating the raw motion data such that the z-axis of each vector is vertically oriented and centered around the mean of 1 g acceleration. In at least one embodiment, the gravitational acceleration 1 g is subtracted from the aligned acceleration data. The aligned motion data includes aligned acceleration data denoted aₚ=[aₓ, a_{y}, a_{z}], aligned orientation data denoted oₚ=[oₓ, o_{y}, o_{z}], and aligned magnetic field data denoted mₚ=[mₓ, m_{y}, m_{z}].

In at least one embodiment, the processor 122 is configured to utilize a quaternion based approach to calculate the aligned motion data {aₚ, oₚ, mₚ}. Particularly, let the quaternion from the 9-DOF IMU 112 be denoted as q. The acceleration data can be represented as another quaternion:

a_{q}=0+aₓi+a_{y}j+a_{z}k.

The processor 122 rotates the acceleration data the quaternion in order to match the world frame according to the following equation:

aₚ=q*a_{q}q,

where aₚ is another quaternion with real part w=0 and is treated as a vector.

In at least one embodiment, the processor 122 calculates the aligned orientation data oₚ and the aligned magnetic field data mₚ by rotating in the same manner. However, it should be appreciated that other techniques for reorienting the motion data to align with the direction of gravity can be utilized in alternative embodiments.

In at least one embodiment, the processor 122 further transforms the orientation of the raw motion data {a, o, m} to align with the direction of magnetic north and/or true north. Particularly, the processor 122 calculates the aligned motion data {aₚ, oₚ, mₚ} by further determining the direction of magnetic north and/or true north based on the raw magnetic field data m and rotating the raw motion data {a, o, m} such that the y-axis of each vector is oriented and centered around the direction of magnetic north and/or true north. It should be appreciated that this is useful for calculating certain types of metadata such as path estimation or indoor localization of the human.

The method 200 continues, in the pre-processing phase, with filtering the motion data with a low-pass filter (block 230). Particularly, the processor 122 determines filtered motion data by filtering at least some of the aligned motion data {aₚ, oₚ, mₚ} with a low pass filter. The processor 122 at least applies a low pass filter to the aligned acceleration data aₚ to determine filtered acceleration data aₚ′=[aₓ, a_{y}, a_{z}]. Since walking is a low-frequency activity by nature, the low pass filtering of the aligned acceleration data aₚ has the effect of eliminating sensor noise and unwanted higher-frequency accelerations. In one embodiment, the low pass filter applied to the aligned acceleration data aₚ is a Butterworth low-pass filter with cut-off frequency of 3 Hz and is applied to each of the three axial components separately.

In at least some embodiments, the processor 122 also applies respective low pass filters to the aligned orientation data oₚ and the aligned magnetic field data mₚ to determine filtered orientation data oₚ′=[oₓ, o_{y}, o_{z}] and filtered magnetic field data mₚ′=[mₓ, m_{y}, m_{z}]. The low pass filtering of the aligned orientation data oₚ and the aligned magnetic field data mₚ, likewise, has the effect of eliminating sensor noise and unwanted higher-frequency changes in orientation and magnetic field.

In at least one embodiment, in the case that there are multiple IMU 112 worn or carried by the human, the processor 122 further determines averaged motion data by averaging the aligned motion data and/or the filtered motion data of each IMU 112. The has the advantage of further reducing sensor noise and further filtering out irrelevant body motions.

The method 200 continues, in the step detection phase, with detecting step regions by detecting peaks and valleys in the vertical accelerations (block 240). Particularly, the processor 122 is configured to detect a plurality of step segments S (also referred to as “step regions”) of the filtered motion data {aₚ′, oₚ′, mₚ′} corresponding to individual steps of a walking activity by detecting peaks and valleys in the filtered acceleration data aₚ′. As used herein, a “segment” or “region” of the motion data refers to a continuous sequence of values of the motion data, e.g., motion data starting from a first index or timestamp and ending at a second index or timestamp that is later in time.

It should be appreciated that a walking step normally follows an acceleration-deceleration pattern, which can be uncovered by peak-valley detection algorithm applied to the accelerometer readings. In at least one embodiment, the processor 122 applies a peak-valley detection algorithm to the vertical (z-axis) component of a_{z} of the filtered acceleration data aₚ′. Advantageously, the vertical accelerations alone provide a more stable signal and contain the least interference from irrelevant motions from the human body. In addition, in the case that there are multiple IMU 112 worn or carried by the human, the vertical accelerations from the multiple IMU 112 are averaged, which further filters out irrelevant body motions.

In at least one embodiment, the processor 122 identifies peaks and valleys of vertical accelerations a_{z} of the filtered acceleration data aₚ′ by, for each individual measurement a_{z}⁽ⁱ⁾ in the time series of vertical accelerations a_{z}, comparing the individual measurement a_{z}⁽ⁱ⁾ with the previous measurement a_{z}⁽ⁱ⁻¹⁾ and with the subsequent measurement a_{z}⁽ⁱ⁺¹⁾, the where i is the index of the respective individual measurement a_{z}⁽ⁱ⁾ under consideration. If an individual measurement a_{z}⁽ⁱ⁾ is greater than both the previous measurement a_{z}⁽ⁱ⁻¹⁾ and the subsequent measurement a_{z}⁽ⁱ⁺¹⁾ (i.e., a_{z}⁽ⁱ⁾>a_{z}⁽ⁱ⁻¹⁾ and a_{z}⁽ⁱ⁾>a_{z}⁽ⁱ⁺¹⁾), then the processor 122 identifies a local peak at the index i. Conversely, if an individual measurement a_{z}⁽ⁱ⁾ is less than both the previous measurement a_{z}⁽ⁱ⁻¹⁾ and the subsequent measurement a_{z}⁽ⁱ⁺¹⁾ (i.e., a_{z}⁽ⁱ⁾<a_{z}⁽ⁱ⁻¹⁾ and a_{z}⁽ⁱ⁾<a_{z}⁽ⁱ⁺¹⁾), then the processor 122 identifies a local valley at the index i. Otherwise, if neither set of conditions is true, then the processor 122 does not identify a peak or valley at the index i.

Next, once the local peaks and local valleys are identified in the time series of vertical accelerations a_{z}, the processor 122 determines the step segments S each as a continuous sequence of values of the motion data starting at a first index of a first local peak in the time series of vertical accelerations a_{z}, ending with a subsequent second index of a second local peak in the time series of vertical accelerations a_{z}, and including an local valley in the time series of vertical accelerations a_{z} between the first local peak and second local peak. Thus, each step segment is formed the adjacent sequence peak-valley-peak.

It should, of course, be appreciated that this peak-valley-peak sequence formulation implies a particular polarity of the vertical accelerations a_{z}. However, in some embodiments, a valley-peak-valley sequence can be equivalently detected. Accordingly, as used herein, a “local peak” in the vertical acceleration data refers to a local maximum acceleration in a particular direction that is axially aligned and/or parallel with the direction of gravity, regardless of the polarity of the data itself. Likewise, as used herein, a “local valley” in the vertical acceleration data refers to a local minimum acceleration in the particular direction.

In some embodiments, the processor 122 only determines peak-valley-peak sequence to be a step segment S if the acceleration gradient between the peaks and the valley exceed a minimum acceleration gradient threshold and if the time duration of the sequence is within a predetermined range. Particularly, the processor 122 forms a step segment only if a respective peak-valley-peak sequence satisfies the following conditions:

a_{z}^{start}−a_{z}^{middle}>T_{grad} and a_{z}^{end}−a_{z}^{middle}>T_{grad},

end−start>Lₘᵢₙ and end−start<Lₘₐₓ,

where start is the index of a first local peak in a_{z}, end is the index of a second local peak in a_{z}, and middle is the index of a local valley between the first local peak and the second local peak. T_{grad} is minimum acceleration gradient threshold (e.g., T_{grad}=0.04), and Lₘᵢₙ, Lₘₐₓ are limits defining an acceptable range of time durations for an individual step segment (e.g., Lₘᵢₙ=0.3 s and Lₘₐₓ=1 s).

In this way, the processor 122 identifies a plurality of step segments S, each corresponding to an individual step of the human. Each step segment Sₙ in the plurality of step segments S includes filtered motion data {aₚ′, oₚ′, mₚ′}^{start}^{ₙ}^{:end}^{ₙ} beginning at a respective index (or timestamp) of the first local peak denoted startₙ and ending at a respective index (or timestamp) of the second local peak denoted endₙ, with a mid-point at a respective index (or timestamp) of the local valley denoted middleₙ, where n is the index of the particular step segment Sₙ among the plurality of step segments S.

The method 200 continues, in the filtering and post-processing phase, with filtering out false-positive step regions based on timing and similarity (block 250). Particularly, the processor 122 evaluates each step segment Sₙ among the plurality of step segments S against at least one criterion in order to determine whether the step segment Sₙ is a false-positive or, in other words, whether the step segment Sₙ does not correspond to an actual step taken by the human. Particularly, it should be appreciated that irrelevant body motions that do not actually correspond to a step taken by the human may nonetheless follow the same peak-valley-peak sequence pattern defined above, thus causing false-positives. Accordingly, it is advantageous to apply a variety of criteria to filter out false-positives from the plurality of step segments S.

In some embodiments, the processor 122 determines whether a step segment Sₙ is a false-positive based on the indices or timestamps startₙ, middleₙ, and endₙ for the step segment Sₙ. Particularly, in one embodiment, the processor 122 determines that a respective step segment Sₙ is a false-positive if there is greater than a threshold time Tₜᵢₘₑ (e.g., Tₜᵢₘₑ=1 s) between the respective step segment Sₙ and both of the adjacent step segments Sₙ₋₁ and Sₙ₊, where the step segment Sₙ₋₁ is the immediately previous in time step segment and step segment Sₙ₊₁ is the immediately subsequent in time step segment. In other words, the processor 122 determines that a respective step segment Sₙ is not a false-positive if at least one of the following criteria is satisfied:

startₙ−endₙ₋₁<Tₜᵢₘₑ or startₙ₊₁−endₙ<Tₜᵢₘₑ,

indicating that the step segment Sₙ is sufficiently close in time to at least one adjacent step segment Sₙ₋₁ or Sₙ₊₁. If the step segment Sₙ is determined to be a false-positive, the processor 122 removes it from the plurality of step segments S. The basis for this criterion is that, in general, steps always appear in groups during walking. Therefore, a step segment Sₙ that is isolated from both adjacent step segments Sₙ₋₁ and Sₙ₊₁ by at least the threshold time Tₜᵢₘₑ is considered a false-positive for the purpose of detecting walking activity.

In some embodiments, the processor 122 determines whether a step segment Sₙ is a false-positive based on the filtered motion data {aₚ′, oₚ′, mₚ′}^{start}^{ₙ}^{:end}^{ₙ}, or more particularly the time series of vertical accelerations a_{z}^{start}^{ₙ}^{:end}^{ₙ} between the indices/timestamps startₙ and endₙ. Particularly, in one embodiment, the processor 122 determines whether a respective step segment Sₙ is a false-positive based on a similarity (or difference) between the time series of vertical accelerations a_{z}^{start}^{ₙ}^{:end}^{ₙ} and those of the adjacent step segments Sₙ₋₁ and Sₙ₊₁ (i.e., a_{z}^{start}^{_{n−1}}^{:end}^{_{n−1}} and a_{z}^{start}^{ₙ₊₁}^{:end}^{ₙ₊₁}).

In at least one embodiment, for the purpose of evaluating similarity (or difference), the processor 122 maps a_{z}^{start}^{ₙ}^{:end}^{ₙ} onto a_{z}^{start}^{_{n−1}}^{:end}^{_{n−1}} (or vice versa) using a mapping algorithm, such as a dynamic time warping algorithm. Likewise, the processor 122 maps a_{z}^{start}^{ₙ}^{:end}^{ₙ} onto a_{z}^{start}^{ₙ₊₁}^{:end}^{ₙ₊₁} (or vice versa) using a mapping algorithm, such as a dynamic time warping algorithm. Next, the processor 122 determines the similarity between a_{z}^{start}^{ₙ}^{:end}^{ₙ} and a^{start}^{_{n−1}}^{:end}^{_{n−1}} as an average geometric distance/difference between a_{z}^{start}^{ₙ}^{:end}^{ₙ} and a_{z}^{start}^{_{n−1}}^{:end}^{_{n−1}} after mapping. Likewise, the processor 122 determines the similarity between a_{z}^{start}^{ₙ}^{:end}^{ₙ} and a_{z}^{start}^{ₙ₊₁}^{:end}^{ₙ₊₁} as an average geometric distance/difference between a_{z}^{start}^{ₙ}^{:end}^{ₙ} and a_{z}^{start}^{ₙ₊₁}^{:end}^{ₙ₊₁} after mapping. In these examples, a smaller average geometric distance/difference indicates a higher level of similarity. It should be appreciated that other distances measures and similarity measures can be similarly utilized.

Once the similarity between the time series of vertical accelerations a_{z}^{start}^{ₙ}^{:end}^{ₙ} and those of the adjacent step segments Sₙ₋₁ and Sₙ₊₁ (i.e., a_{z}^{start}^{_{n−1}}^{:end}^{_{n−1}} and a_{z}^{start}^{ₙ₊₁}^{:end}^{ₙ₊₁}) the processor 122 determines that a respective step segment Sₙ is a false-positive if there is greater than a threshold distance T_{dist} (e.g., T_{dist}=0.008) (or less than a threshold similarity) between the respective step segment Sₙ and one or both of the adjacent step segments Sₙ₋₁ and Sₙ₊₁. In other words, the processor 122 determines that a respective step segment Sₙ is a false-positive if one or both of the following criteria are violated:

dist(a_{z}^{start}^{ₙ}^{:end}^{ₙ}, a_{z}^{start}^{_{n−1}}^{:end}^{_{n−1}})<T_{dist} and

dist(a_{z}^{start}^{ₙ}^{:end}^{ₙ},a_{z}^{start}^{ₙ₊₁}^{:end}^{ₙ₊₁})<T_{dist},

where dist( ) is a distance function or other difference function in which a smaller value indicates a higher level of similarity. It some embodiments, a respective step segment Sₙ is considered a false-positive if either one of the criteria are violated (i.e., the step segment Sₙ is dissimilar from either one of the adjacent step segments Sₙ₋₁ and Sₙ₊₁). Alternatively, in other embodiments, a respective step segment Sₙ is considered a false-positive only if both of the criteria are violated (i.e., the step segment Sₙ is dissimilar from both of the adjacent step segments Sₙ₋₁ and Sₙ₊₁). If the step segment Sₙ is determined to be a false-positive, the processor 122 removes it from the plurality of step segments S.

The method 200 continues, in the filtering and post-processing phase, with forming walking regions by merging step regions (block 260). Particularly, the processor 122 forms a plurality of walking segments W (also referred to as “walking regions”) of the filtered motion data {aₚ′, oₚ′, mₚ′} corresponding to individual continuous periods of walking by merging groups of adjacent step segments in the plurality of step segments S.

Particularly, the processor 122 identifies groups of adjacent step segments in the plurality of step segments S in which each step segment in a respective group is within a threshold time T_{merge} (e.g., T_{merge}=1 s) from immediately adjacent step segments in the respective group. In other words, no step segment in a respect group is more than the threshold time T_{merge} from immediately adjacent step segments in the respective group. The processor 122 merges each identified group of adjacent step segments to form a respective one of the plurality of walking segments W.

Thus, the processor 122 defines a plurality of walking segments W, each corresponding to individual continuous periods of walking. Each step segment Wₘ in the plurality of walking segments W includes filtered motion data {aₚ′, oₚ′, mₚ}^{start}^{ₘ}^{:end}^{ₘ} beginning at a respective starting index (or starting timestamp), denoted startₘ, of the first-in-time step segment of the respective group of step segments that formed the step segment Wₘ and ending at a respective ending index (or ending timestamp), denoted endₘ, of the last-in-time step segment of the respective group of step segments that formed the step segment Wₘ, where m is the index of the particular walking segment Wₘ among the plurality of walking segments W.

The method 200 continues, in the filtering and post-processing phase, with calculating magnitudes of the horizontal accelerations (block 270). Particularly, the processor 12 is configured to calculate a time series of horizontal acceleration magnitudes aₕₒᵣ, which are orthogonal to the direction of gravity, based the horizontal acceleration components aₓ, a_{y} of the filtered acceleration data aₚ′. The processor 122 at least calculates horizontal acceleration magnitudes aₕₒᵣ for portions of the filtered acceleration data aₚ′ that are included in one of plurality of walking segments W, but may simply calculate horizontal acceleration magnitudes aₕₒᵣ for all of the filtered acceleration data aₚ′. In at least one embodiment, time series of the horizontal acceleration magnitudes aₕₒᵣ is calculated according to the formula:

aₕₒᵣ=\sqrt{aₓ²+a_{y}²}.

The method 200 continues, in the filtering and post-processing phase, with filtering out false-positive walking regions based on variation in the magnitudes of the horizontal accelerations (block 280). Particularly, the processor 122 evaluates each walking segment Wₘ among the plurality of walking segments W against at least one criterion in order to determine whether the walking segment Wₘ is a false-positive or, in other words, whether the walking segment Wₘ does not correspond to a period of continuous walking by the human.

In some embodiments, the processor 122 determines whether a walking segment Wₘ is a false-positive based on the horizontal acceleration magnitudes aₕₒᵣ or more particularly the time series of horizontal acceleration magnitudes aₕₒᵣ^{start}^{ₘ}^{:end}^{ₘ} between the indices/timestamps startₘ and endₘ. In at least one embodiment, the processor 122 determines a variation metric, in particular either a variance or a standard deviation, of aₕₒᵣ^{start}^{ₘ}^{:end}^{ₘ}. If the variation metric is less than a threshold T_{std} (e.g., T_{std}=0.06), then the processor 122 determines that the walking segment Wₘ is a false positive and removes it from the plurality of walking segments W. In this way, only walking segments that include some horizontal displacement of the body are counted (i.e., walking in place or similar motions are ignored).

In some embodiments, once a final set of walking segments W have been identified, the processor 122 writes metadata of the motion data to the memory 124 that indicates start and end timestamps (i.e., startₘ and endₘ) for each continuous period of walking (i.e., each walking segment Wₘ) in the motion data. These timestamps (which may also be referred to as “labels” of the motion data) can be used to perform further processing to determine additional secondary metadata based on the labeled periods of continuous walking in the motion data. Such secondary metadata may include, for example, a step count indicating a total number of steps taken in during some interval of time, a path estimation indicating a path taken by the human during a walking activity for some interval of time, a metric describing a gait of the human (e.g., stride length, etc.), and indoor localization information indicating an estimated position of the human within an indoor environment. It should be appreciated that a wide variety of secondary metadata can be determined on the basis of the motion data with the labeled periods of continuous walking.

Embodiments within the scope of the disclosure may also include non-transitory computer-readable storage media or machine-readable medium for carrying or having computer-executable instructions (also referred to as program instructions) or data structures stored thereon. Such non-transitory computer-readable storage media or machine-readable medium may be any available media that can be accessed by a general purpose or special purpose computer. By way of example, and not limitation, such non-transitory computer-readable storage media or machine-readable medium can comprise RAM, ROM, EEPROM, CD-ROM or other optical disk storage, magnetic disk storage or other magnetic storage devices, or any other medium which can be used to carry or store desired program code means in the form of computer-executable instructions or data structures. Combinations of the above should also be included within the scope of the non-transitory computer-readable storage media or machine-readable medium.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, objects, components, and data structures, etc. that perform particular tasks or implement particular abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

While the disclosure has been illustrated and described in detail in the drawings and foregoing description, the same should be considered as illustrative and not restrictive in character. It is understood that only the preferred embodiments have been presented and that all changes, modifications and further applications that come within the spirit of the disclosure are desired to be protected.

What is claimed is:

1. A method for recognizing a walking activity, the method comprising:
receiving, with a processor, motion data at least including a time series of acceleration values corresponding to motions of a human that include walking;
defining, with the processor, a first plurality of segments of the received motion data by detecting local peaks and local valleys in the time series of acceleration values, each segment in the first plurality of segments including respective motion data corresponding to an individual step of the human; and
defining, with the processor, a second plurality of segments of the received motion data by merging each of a plurality of groups of segments in the first plurality of segments, each segment in the second plurality of segments including respective motion data corresponding to a period of continuous walking by human.

2. The method according to claim 1, wherein each value in the time series of acceleration values is a three-dimensional acceleration value, the method further comprising:
transforming, with the processor, an orientation of the time series of acceleration values such that a first axis of each three-dimensional acceleration value is aligned with a direction of gravity.

3. The method according to claim 2, the defining the first plurality of segments further comprising:
detecting, with the processor, a plurality of local peaks and a plurality of local valleys in a time series of vertical acceleration values, which are first axis components of the three-dimensional acceleration values of the time series of acceleration values.

4. The method according to claim 3, the defining the first plurality of segments further comprising:
defining, with the processor, each respective segment of the first plurality of segments to include the respective motion data including a respective time series of acceleration values that (i) starts with a respective first local peak of the plurality of local peaks, (ii) ends with a respective second local peak of the plurality of local peaks, and (iii) includes a respective local valley of the plurality of local valleys situated in time between the respective first local peak and the respective second local peak.

5. The method according to claim 4, the defining the first plurality of segments further comprising:
defining, with the processor, each respective segment of the first plurality of segments only if (i) a difference between acceleration values of the respective first local peak and the respective local valley exceed a predetermined acceleration threshold and (ii) a difference between acceleration values of the respective second local peak and the respective local valley exceed the predetermined acceleration threshold.

6. The method according to claim 4, the defining the first plurality of segments further comprising:
defining, with the processor, each respective segment of the first plurality of segments only if a difference between time values of the respective first local peak and the respective second local peak is within a predetermined range.

7. The method according to claim 4 further comprising, for each respective segment of the first plurality of segments:
removing, with the processor, the respective segment from the first plurality of segments in response to (i) a difference between a start time of the respective segment and an end time an adjacent previous-in-time segment of the first plurality of segments being greater than a threshold time, and (ii) a difference between an end time of the respective segment and a start time an adjacent subsequent-in-time segment of the first plurality of segments being greater than the threshold time.

8. The method according to claim 4 further comprising, for each respective segment of the first plurality of segments:
determining, with the processor, similarities (i) between the time series of acceleration values of the respective segment and the time series of acceleration values of an adjacent previous-in-time segment of the first plurality of segments and (ii) between the time series of acceleration values of the respective segment and time series of acceleration values of an adjacent subsequent-in-time segment of the first plurality of segments; and
removing, with the processor, the respective segment from the first plurality of segments in response to the time series of acceleration values of the respective segment having less than a threshold similarity to at least one of (i) the time series of acceleration values of the adjacent previous-in-time segment and (ii) the time series of acceleration values of the adjacent subsequent-in-time segment.

9. The method according to claim 8, the determining the similarities further comprising:
mapping, with the processor, the time series of acceleration values of the respective segment onto the time series of acceleration values of the adjacent previous-in-time segment; and
mapping, with the processor, the time series of acceleration values of the respective segment onto the time series of acceleration values of the adjacent subsequent-in-time segment.

10. The method according to claim 9, the determining the similarities further comprising:
determining, with the processor, a first average geometric distance between the time series of acceleration values of the respective segment and the time series of acceleration values of the adjacent previous-in-time segment, after the mapping thereof; and
determining, with the processor, a second average geometric distance between the time series of acceleration values of the respective segment and the time series of acceleration values of the adjacent subsequent-in-time segment, after the mapping thereof.

11. The method according to claim 2, the defining the second plurality of segments further comprising:
identifying, with the processor, each respective group of the plurality of groups such every segment the respective is within a predetermined threshold time of at least one adjacent segment in the respective group.

12. The method according to claim 11, the defining the second plurality of segments further comprising:
defining, with the processor, each respective segment of the second plurality of segments to include the respective motion data including a respective time series of acceleration values that (i) starts with a start of a first-in-time segment of a respective group of the plurality of groups and (ii) ends with an end of a last-in-time segment of the respective group.

13. The method according to claim 12, further comprising, for each respective segment of the second plurality of segments:
determining, with the processor, a respective time series of horizontal acceleration values based on second axis components and third axis components of three-dimensional acceleration values of the respective time series of acceleration values, which are orthogonal to the first axis that is aligned with the direction of gravity.

14. The method according to claim 13, further comprising, for each respective segment of the second plurality of segments:
determining, with the processor, a respective variation metric of the respective time series of horizontal acceleration values, the respective variation metric being one of a variance and a standard deviation; and
removing, with the processor, the respective segment from the second plurality of segments in response to the respective variation metric being less than a predetermined variation threshold.

15. The method according to claim 1 further comprising:
filtering, with a low pass filter, the time series of acceleration values before identifying the first plurality of segments.

16. The method according to claim 1 further comprising:
determining, with the processor, based on the second plurality of segments of the received motion data, at least one of a step count of the human, a path taken by the human, a metric describing a gait of the human, and a localization of the human.

17. A system for recognizing a walking activity, the system comprising:
at least one motion sensor configured to capture motion data at least including a time series of acceleration values corresponding to motions of a human that include walking; and
a processing system having at least one processor configured to:
receive the motion data from the motion sensor;
define a first plurality of segments of the received motion data by detecting local peaks and local valleys in the time series of acceleration values, each segment in the first plurality of segments including respective motion data corresponding to an individual step of the human; and
define a second plurality of segments of the received motion data by merging each of a plurality of groups of segments in the first plurality of segments, each segment in the second plurality of segments including respective motion data corresponding to a period of continuous walking by human.

18. A non-transitory computer-readable medium for recognizing a walking activity, the computer-readable medium storing program instructions that, when executed by a processor, cause the processor to:
receive motion data at least including a time series of acceleration values corresponding to motions of a human that include walking;
define a first plurality of segments of the received motion data by detecting local peaks and local valleys in the time series of acceleration values, each segment in the first plurality of segments including respective motion data corresponding to an individual step of the human; and
define a second plurality of segments of the received motion data by merging each of a plurality of groups of segments in the first plurality of segments, each segment in the second plurality of segments including respective motion data corresponding to a period of continuous walking by human.
